(19) United States
(12) Patent Application Publication
(10) Pub. No.: US 2026/0208161 A1
(43) Pub. Date: Jul. 23, 2026

(54) CATALYST FOR PARAFFIN DEHYDROGENATION

(71) Applicant: SABIC GLOBAL TECHNOLOGIES B.V., Bergen op Zoom (NL)

(72) Inventors: Biju Maippan DEVASSY, Bangalore (IN); Vinod Sankaran NAIR, Bangalore (IN); Velayutham SARAVANAN, Hosur (IN); Nigit Jose MELEPPURAM, Bangalore (IN); Ritesh NANDY, Bangalore (IN)

(21) Appl. No.: 19/140,923

(22) PCT Filed: Dec. 18, 2023
(86) PCT No.: PCT/EP2023/086284

§ 371 Date: Jun. 18, 2025

Related U.S. Application Data
(60) Provisional application No. 63/476,643, filed on Dec. 22, 2022.

(51) Int. Cl. B01J 21/02; B01J 6/00; B01J 23/26; B01J 35/80; B01J 37/04; B01J 38/10; B01J 38/12; C07C 5/333

(52) U.S. Cl. CPC B01J 21/02; B01J 6/001; B01J 23/26; B01J 35/80; B01J 37/04; B01J 38/10; B01J 38/12; C07C 5/3332; C07C 2521/02; C07C 2523/26

(57) ABSTRACT

Methods of preparing and using alkane dehydrogenation catalysts are provided. The method for making an alkane dehydrogenation catalyst may include mixing a plurality of aluminum hydroxides, a chromium (III) oxide source, and an alkali metal oxide source with an aqueous metal free acid solution to form a moldable mixture. The plurality of aluminum hydroxides may contain from about 60 wt. % to about 99 wt. % of crystalline aluminum trihydroxide and from about 1 wt. % to about 40 wt. % of crystalline aluminum oxide-hydroxide or gelatinous aluminum hydroxide, or combinations thereof. The method may further include extruding the moldable mixture to form extrudates and drying and calcining the extrudates to produce the alkane dehydrogenation catalyst.

### CROSS-REFERENCE TO RELATED APPLICATIONS

This application claims the benefit of priority to U.S. Provisional Patent Application No. 63/476,643, filed Dec. 22, 2022, the entire contents of which are hereby incorporated by reference in their entirety.

### TECHNICAL FIELD

The present invention generally relates to methods for preparing and using an alkane dehydrogenation catalyst. More specifically, the present invention relates to, among other embodiments, methods for the use and preparation of chromia-alumina dehydrogenation catalysts without the use of water-soluble chromium containing materials for the dehydrogenation of paraffins.

### BACKGROUND

Alkane dehydrogenation is a recognized process for the production of a variety of useful hydrocarbon products, such as isobutylene for conversion to MTBE, as well as isooctane and alkylates to supplement and enrich gasolines. There are several current catalytic processes useful for the catalytic dehydrogenation of light alkanes, including the Sud-Chemie CATOFIN® process, UOP's Oleflex® process, Phillips' Star™ process, and the Snamprogetti-Yarsintee process. The catalysts that are used in these processes are manufactured from two different groups of materials. The Sud-Chemie CATOFIN® process and the Snamprogetti-Yarsintee process utilize chromia-alumina catalysts. In contrast, the catalysts for the UOP and Phillips processes include supported precious metal platinum as catalysts.

Chromia-alumina dehydrogenation catalyst technology has been in use for many decades. The stability of dehydrogenation catalysts plays an important role in the overall efficiency of the dehydrogenation process. Because of the extreme temperature ranges at which the catalytic dehydrogenation procedure is conducted, the life expectancy of the catalyst is often limited. Thus, improving the stability of the catalyst translates into longer catalyst life, allowing for better catalyst utilization and ultimately resulting in lower consumption of the catalyst during the dehydrogenation process. One of the important requirements in the production of pelleted/extruded catalysts for use in fixed-bed catalytic operations is that the catalyst be of sufficient strength to support its own weight as well as the process-stream turbulence and similar disturbances ordinarily encountered in fixed-bed processes. Failure to satisfy these requirements is a frequent source of difficulty. Additionally, the gradual loss of mechanical strength during use is a common occurrence necessitating the discarding of catalyst which in other respects would continue to be entirely satisfactory.

There are a number of different types of alumina that are available for use as the support for dehydrogenation catalysts. However, mid to high surface area gamma alumina has consistently been the preferred choice as the carrier for such catalysts as disclosed, for example, in U.S. Pat. Nos. 2,956,030, 2,945,823 and 2,374,404. It is reported that chromium (III) oxide supported on eta-alumina is highly stable compared to chromia supported on gamma-alumina (PHYSICAL REVIEW B 2003, 67, 115414; Adv. Mater. 2007, 19, 2129-2133; Energy Technol. 10.1002/ente.201800736).

Current chromia-alumina dehydrogenation catalysts are produced by the impregnation of an aluminum carrier with a high-concentration chromic acid solution, which contains primarily hexavalent chromium (chromium(VI)), which is toxic and carcinogenic, and therefore highly undesirable for use on an industrial scale. Attempts to overcome the use of hexavalent chromium have been described. For example, CN Patent No. 101940922B discloses making a Cr₂O₃/alumina carrier and then immersing the carrier with additional chromium nitrate and potassium nitrate. U.S. patent Ser. No. 10/646,853 to Fridman also discloses making a Cr₂O₃/alumina carrier and then impregnating the carrier with an aqueous solution containing chromium nitrate, sodium hydroxide, magnesium nitrate, and zirconium carbonate. These methods suffer from the fact that catalyst preparation involves an impregnation process followed by drying and calcination steps, which are time and labor intensive as well as costly. CN Patent No. 102794167A describes an attempt to produce a chromium based dehydrogenation catalyst that includes extruding an acidic solution of chromium (III) oxide powder, activated alumina powder, calcium nitrate and potassium nitrate, followed by drying and calcining. The dried extrudates are then baked at 540° C. for 4 hours with nitrogen gas to obtain the catalyst. A catalyst formed with this process is expected to show alumina in gamma alumina form which strongly affects catalyst stability. Additionally, the catalyst preparation process involving the impregnation of an aluminum carrier with a chromium (III) salt requires multiple impregnation steps to arrive at a desirable chromium content. Each impregnation step requires intermediate drying and calcination, and so such a multi-impregnation method is time- and labor-intensive, and cost prohibitive relative to conventional preparation procedures involving chromium(VI)-containing materials.

Accordingly, the inventors have recognized that there exists a need for a simple, cost effective method for making a chromia-alumina dehydrogenation catalyst without the use of chromium (VI) containing materials, and which exhibits good activity, improved stability, and suitable mechanical strength for the dehydrogenation of lower paraffins.

### SUMMARY

To address shortcomings in the art, the Applicant has developed methods for the preparation and use of alkane dehydrogenation catalyst. In addition to other embodiments, the Applicant has discovered cost-effective methods for making chromia-alumina dehydrogenation catalysts without the use of water soluble chromium containing sources. The presently disclosed dehydrogenation catalysts have good activity as well as improved stability and mechanical strength. In certain embodiments of the presently disclosed method of preparing a dehydrogenation catalyst, impregnation of an aluminum carrier with a chromium (III) salt may not be required or included. In certain embodiments, the presently disclosed method of preparing a dehydrogenation catalyst excludes an impregnation step. The presently disclosed chromia-alumina dehydrogenation catalysts are useful, among other uses, for the dehydrogenation of paraffins.

According to one aspect of the present disclosure, a method for making an alkane dehydrogenation catalyst is provided. In certain embodiments, the method may include mixing a plurality of aluminum hydroxides, a water insoluble chromium (III) oxide source, and an alkali metal oxide source with an aqueous metal free acid solution to form a moldable mixture. The plurality of aluminum hydroxides may contain from about 60 weight percent (wt. %) to about 99 wt. % of crystalline aluminum trihydroxide and from about 1 wt. % to about 40 wt. % of crystalline aluminum oxide-hydroxide or gelatinous aluminum hydroxide, or combinations thereof, or preferably from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof, and more preferably from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof. In certain embodiments, the plurality of aluminum hydroxides may contain a combination of from about 90 wt. % to about 99 wt. % of crystalline aluminum trihydroxide and from about 1 wt. % to about 10 wt. % of crystalline aluminum oxide-hydroxide or gelatinous aluminum hydroxide. The method may further include extruding the moldable mixture to form extrudates and drying and calcining the extrudates to produce the alkane dehydrogenation catalyst.

According to another aspect of the present disclosure, a method for dehydrogenation of an alkane is provided. In certain embodiments, the method may include loading a reactor with a dehydrogenation catalyst produced by mixing a plurality of aluminum hydroxides, a water insoluble chromium (III) oxide source, and an alkali metal oxide source with an aqueous metal free acid solution to form a moldable mixture. The plurality of aluminum hydroxides may contain from about 60 wt. % to about 97 wt. % of crystalline aluminum trihydroxide and from about 3 wt. % to about 40 wt. % of crystalline aluminum oxide-hydroxide or gelatinous aluminum hydroxide. In certain embodiments, the plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide.

The method for dehydrogenation of an alkane may further include extruding the moldable mixture to form extrudates and drying and calcining the extrudates to produce the alkane dehydrogenation catalyst. The method may further include supplying a feed containing alkanes through the reactor at a temperature sufficient to dehydrogenate the alkanes. In certain embodiments, the method may further include separating a dehydrogenated product from unreacted alkanes.

Still other aspects and advantages of these exemplary embodiments and other embodiments are discussed in detail herein. Moreover, it is to be understood that both the foregoing information and the following detailed description provide merely illustrative examples of various aspects and embodiments and are intended to provide an overview or framework for understanding the nature and character of the claimed aspects and embodiments. Accordingly, these and other objects, along with advantages and features of the present disclosure, will become apparent through reference to the following description. Furthermore, it is to be understood that the features of the various embodiments described herein are not mutually exclusive and may exist in various combinations and permutations.

### DETAILED DESCRIPTION

The present disclosure describes various embodiments related to methods for preparing and using an alkane dehydrogenation catalyst. Further embodiments may be described and disclosed.

In the following description, numerous details are set forth in order to provide a thorough understanding of the various embodiments. In other instances, well-known processes, devices, and systems may not have been described in particular detail to not unnecessarily obscure the various embodiments. Additionally, illustrations of the various embodiments may omit features or details to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” is defined as being close to as understood by one of ordinary skill in the art. In one non-limiting embodiment, the terms are defined to be within 10%, preferably within 5%, more preferably within 1%, and most preferably within 0.5%.

The use of the words “a” or “an” when used with any of the terms “comprising,” “including,” “containing,” or “having,” in the claims or the specification may mean “one,” but it is also consistent with the meaning of “one or more,” “at least one,” and “one or more than one.”

The terms “wt. %”, “vol. %”, or “mol. %” refer to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “substantially free” of a component X refers to a composition that contains no more than 1 wt. % of the component X in the composition.

Disclosed here are methods for preparing and using an alkane dehydrogenation catalyst. The dehydrogenation catalysts prepared according to the presently disclosed methods have good activity as well as improved stability and mechanical strength. The presently disclosed methods provide cost-effective methods for making chromia-alumina dehydrogenation catalysts without the use of chromium(VI) containing sources. The presently disclosed methods provide cost-effective methods for making chromia-alumina dehydrogenation catalysts without the use of water soluble chromium containing sources. In certain embodiments, the presently disclosed method of preparing a dehydrogenation catalyst does not require or include impregnation of an aluminum carrier with a chromium (III) salt. In certain embodiments, the presently disclosed method of preparing a dehydrogenation catalyst excludes an impregnation step. The presently disclosed chromia-alumina dehydrogenation catalysts are useful, among other uses, for the dehydrogenation of paraffins for the production of olefins. Examples of the paraffins include propane, isobutane, n-butane and isopentane.

According to one aspect of the present disclosure, a method for making an alkane dehydrogenation catalyst is provided. In certain embodiments, the method may include mixing a plurality of aluminum hydroxides, a water insoluble chromium (III) oxide source, and an alkali metal oxide source with an aqueous metal free acid solution to form a moldable mixture. The alkali metal oxide sources can contain a sodium oxide, lithium oxide, potassium oxide, or cesium oxide source. The various components can be mixed by a variety of methods, both manual and mechanical, to provide the moldable mixture. For example, In certain embodiments, the moldable mixture may be mixed by a batch mixer. The raw materials used for the catalyst preparation can be mixed well in a high shear mixer followed by mixing with a metal free aqueous acid solution until a rather stiff dough/granules are obtained. This dough/granules can be extruded and/or formed into any suitable shape including cylinders, cubes, stars, tri-lobes, quadra-lobes, pellets, pills, or spheres by suitable mechanical means. In one embodiment, mixing is conducted in a high intensity environment, such as that supplied by a B&P Littleford Mixer available from B&P Littleford, 1000 Hess Avenue, Saginaw, MI 48601.

In another embodiment, mixing is conducted using an Eirich Intensive Mixer, such as that supplied by Maschinenfabrik GustavEirich Gmbh & Co KG, Hardheim, Germany. Mixing is conducted for a time sufficient to result in a uniform mixture. In other embodiments, other batch or continuous processes can be used to create the moldable mixture. Components may be added serially or together in any convenient order, as would be apparent to the person of ordinary skill in the art.

The plurality of aluminum hydroxides may contain from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof. In certain embodiments, the plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide, or combinations thereof. The plurality of aluminum hydroxides may contain from about 91 wt. % to about 96 wt. % of crystalline aluminum trihydroxide and from about 4 wt. % to about 9 wt. % of crystalline aluminum oxide-hydroxide or gelatinous aluminum hydroxide, or combinations thereof. The plurality of aluminum hydroxides may contain from about 92 wt. % to about 96 wt. % of crystalline aluminum trihydroxide and from about 4 wt. % to about 8 wt. % of crystalline aluminum oxide-hydroxide or gelatinous aluminum hydroxide, or combinations thereof. The plurality of aluminum hydroxides may contain from about 93 wt. % to about 97 wt. % of crystalline aluminum trihydroxide and from about 3 wt. % to about 6 wt. % of crystalline aluminum oxide-hydroxide or gelatinous aluminum hydroxide, or combinations thereof. The plurality of aluminum hydroxides may contain about 95 wt. % of crystalline aluminum trihydroxide and about 5 wt. % of crystalline aluminum oxide-hydroxide or gelatinous aluminum hydroxide, or combinations thereof. The use of a plurality of aluminum hydroxides is expected to increase crush strength without affecting the catalyst performance up to a particular combination of aluminum hydroxides. The method may further include extruding the moldable mixture to form extrudates and drying and calcining the extrudates to produce the alkane dehydrogenation catalyst. In certain embodiments of the method for making an alkane dehydrogenation catalyst, the extrudates may be dried to remove water by heating at a temperature of 50° C. to 200° C., 100° C. to 140° C., 110° C. to 120° C., or 50° C., 55° C., 60° C., 65° C., 70° C., 75° C., 80° C., 85° C., 90° C., 95° C., 100° C., 105° C., 110° C., 115° C., 120° C., 125, ° C., 130° C., 135° C., 140° C., 145° C., 150° C., or any range or value therebetween.

In certain embodiments of the method for making an alkane dehydrogenation catalyst, the extrudates may be calcined at a temperature ranging from about 700° C. to about 1000° C. In certain embodiments, the presently described alkane dehydrogenation catalyst may be in the form of extrudates having a diameter of from about 1 to about 4 millimeters, or from about 2 to about 3.5 millimeters, and a length of from about 2 to about 10 millimeters, or from about 3 to about 9 millimeters.

In certain embodiments, the alkane dehydrogenation catalyst produced by the method may contain from about 60 wt. % to about 90 wt. % of alumina, from about 10 wt. % to about 40 wt. % of water insoluble chromium (III) oxide, and from about 0.1 wt. % to about 5 wt. % of the alkali metal oxide. In certain embodiments, the aqueous metal free acid solution includes acids, such as nitric acid. The moldable mixture includes an aqueous non-metal acid. The non-metal acid herein refers to an acid in which the molecular structure of the acid does not involve a metal atom. In certain embodiments of the process, as otherwise described herein, the non-metal acid may be nitric acid. In other embodiments of the process, as otherwise described herein, the non-metal acid may be an organic acid, such as formic acid or acetic acid. In still other embodiments of the process, the non-metal acid may be a combination of nitric acid and an organic acid such as formic acid or acetic acid. In certain embodiments, the use of an organic acid can be beneficial in that it can reduce the nitrogen oxides concentration during heat treatment. However, it can also make the peptization of aluminum hydroxide less efficient. The person of ordinary skill in the art will determine the appropriate amounts and types of acids to use to provide a desired moldable material.

In certain embodiments, the crystalline aluminum trihydroxide may be selected from one or more of bayerite and nordstrandite. The crystalline aluminum oxide-hydroxides may be boehmite in at least some instances. In certain embodiments, the gelatinous aluminum hydroxides may be selected from one or more of amorphous aluminum hydroxide or pseudoboehmite. In certain embodiments, the water insoluble chromium (III) oxide sources for the making of the alkane dehydrogenation catalyst may be substantially free of hexavalent chromium oxide.

According to another aspect of the present disclosure, a method for dehydrogenation of an alkane is provided. In certain embodiments, the method may include loading a reactor with a dehydrogenation catalyst produced by mixing a plurality of aluminum hydroxides, a chromium (III) oxide source, and an alkali metal oxide source with an aqueous metal free acid solution to form a moldable mixture. The plurality of aluminum hydroxides may contain from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof. In certain embodiments, the plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide, or combinations thereof.

The method for dehydrogenation of an alkane may further include extruding the moldable mixture to form extrudates and drying and calcining the extrudates to produce the alkane dehydrogenation catalyst. The method may further include supplying a feed containing alkanes through the reactor at a temperature sufficient to dehydrogenate the alkanes. In certain embodiments, the method may further include separating a dehydrogenated product from unreacted alkanes. In certain embodiments, the alkane dehydrogenation catalyst used in the method contains from about 60 wt. % to about 95 wt. % of alumina, from about 4 wt. % to about 39 wt. % of chromium (III) oxide, and from about 0.1 wt. % to about 5 wt. % of the alkali metal oxide. In certain embodiments, the alkane dehydrogenation catalyst used in the method contains from about 60 wt. % to about 90 wt. % of alumina, from about 10 wt. % to about 40 wt. % of chromium (III) oxide, and from about 0.1 wt. % to about 5 wt. % of the alkali metal oxide. In some instances, the crystalline aluminum trihydroxide may be selected from one or more of bayerite and nordstrandite. The crystalline aluminum oxide-hydroxides may be boehmite in at least some instances. In certain embodiments, the gelatinous aluminum hydroxides may be selected from one or more of amorphous aluminum hydroxide or pseudoboehmite. In certain embodiments of the method for dehydrogenation of an alkane, the extrudates may be calcined at a temperature ranging from about 700° C. to about 1000° C. The temperature sufficient to dehydrogenate alkanes may range from about 400° C. to 800° C. in at least some of the embodiments of the method.

### EXAMPLES

### Catalyst Preparation

### Example 1 (Comparison Example Prepared without Pseudo-Boehmite)

The catalyst of this example was prepared by mixing Bayerite (2323.6 g, Versal B, UOP), chromium (III) oxide (388.8 g, Sigma-Aldrich®), zirconium (IV) basic carbonate (ZrO)₂(OH)₂CO₃ (17.0 g, Sigma-Aldrich®), which were dry mixed for 10 minutes in an Eirich mixer (EL-5 Profi Plus). An aqueous solution of nitric acid (320 ml, 25 wt. %) containing dissolved sodium nitrate (32.0 g) and magnesium nitrate hexahydrate (123.7 g) was added to the mixer and mixed for about 10 minutes. The obtained blend was aged at 25° C. for 1 hour and then formed into cylindrical extrudates (3.5 mm diameter) using an ETP1 Bonnot lab extruder. The cylindrical extrudates were dried at 70° C. followed by 120° C. for 12 hours, calcined at 850° C. for 2 hours in air in a muffle furnace, cooled to room temperature and used for catalyst testing. The resultant catalyst had a composition of 20 wt. % Cr₂O₃, 0.60 wt. % Na₂O, 1.0 wt. % MgO, 0.7 wt. % ZrO₂ and 77.70 wt. % Al₂O₃.

### Example 2 (Invention with Pseudo-Boehmite)

The catalyst of this example was prepared by dry mixing Bayerite (2101.0 g, Versal B, UOP), pseudo-boehmite (219.9 g, Versal 250, UOP), chromium (III) oxide (390.6 g, Sigma-Aldrich®), zirconium (IV) and basic carbonate (17.1 g, Sigma-Aldrich®) for 10 minutes in an Eirich mixer (EL-5 Profi Plus). An aqueous solution of nitric acid (320 ml of 25 wt. % nitric acid mixed with 30 ml of water) containing dissolved sodium nitrate (32.1 g) and magnesium nitrate hexahydrate (124.2 g) was added to the mixer and mixed for about 10 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 850° C. for 2 hours in air in a muffle furnace, cooled to room temperature and used for catalyst testing. The resultant catalyst of this example had a composition of 20 wt. % Cr₂O₃, 0.60 wt. % Na₂O, 1.0 wt. % MgO, 0.7 wt. % ZrO₂ and 77.70 wt. % Al₂O₃.

### Crush Strength Measurements

The catalyst extrudate crush strength was measured by ASTM D6175 method using Vinci Technologies VERSATILE CATALYST CRUSHING STRENGTH TESTER (VCS). A sample of 50 to 100 g was heated at 400±15° C. for 3 h. After heating, the test sample was cooled in a desiccator to prevent the adsorption of moisture before testing. Only the number of extrudates that could be tested within a 10-min period were removed from the desiccator. The length of the extrudates were measured to the nearest tenth of a millimeter and recorded. Tweezers, forceps, or other suitable devices or procedures were used to prevent the transfer of moisture from the operator's hands to the extrudate being tested. The measured extrudates that had a length to diameter ratio greater than or equal to one, preferably extrudates with length in the range of 6 to 8 mm and diameter in the range of 2.9 to 3.3 mm were placed between the anvils of the compression testing device. The extrudates were positioned flat against the face of the anvil and crushed radially by applying an increasing force at a uniform rate in the range of 1 to 5 lbf/s (4.4 to 22 N/s) until the extrudate crushed or collapsed. The anvils were then separated and all residue was removed with a soft brush. Care was taken to ensure that the faces of the anvils were free from particles adhering to the surface. Each extrudate was subjected to an increasing load up to breaking point and the (maximum load value 100 daN). The compression strength over length ratio is reported as daN/mm. The results are shown in Table 1.

The results provided in Table 1 demonstrate that the catalyst prepared according to the presently disclosed method (Example 2) is characterized by higher crush strength compared to the catalyst prepared by the comparative method (Example 1).

TABLE 1
Crush Strength Measurement Results

| Crush strength | | | | |
|---|---|---|---|---|
| (daN/mm) | Min | Average | Max | STDEV |
| Example 1 | 1.04 | 2.54 | 3.79 | 0.75 |
| Example 2 | 1.65 | 2.67 | 4.00 | 0.66 |

Number of extrudates = 30

### Catalyst Activity Testing

The isobutane dehydrogenation activity of the catalysts of Examples 1 and 2 were tested using a fixed bed reactor. The catalyst loading and reactor details were as follows: Catalyst weight was 70 g, catalyst particle size was about 3 mm diameter, catalyst diluent quartz (ring) size was 2.2×2 mm, catalyst diluent weight ratio was 1:3, reactor inner diameter was 41 mm, reactor outer diameter was 45 mm. The catalyst extrudates (10 g of 7 batches) and inert quartz diluent (30 g of 7 batches) were loaded into reactor in layered manner. Quartz rings mentioned above were loaded above the catalyst bed. A nitrogen purge was employed between the steps of dehydrogenation, catalyst regeneration/oxidation and reduction with hydrogen. The isobutane flow in the dehydrogenation step corresponds to GHSV of 600 ml h⁻¹g⁻¹. The reactor was operated at atmospheric pressure using isobutane (99.9 vol. %) diluted with nitrogen. The reaction pressure during dehydrogenation was 0.33 atmosphere of isobutane and 0.67 atmosphere of nitrogen. The reactor outlet gases were analyzed by online gas chromatograph (Agilent 6890) equipped with a flame ionization detector for hydrocarbon analysis and a thermal conductivity detector for hydrogen analysis. The reactant and products flow rates were measured using a Ritter type wet gas flow meter. The reactor was operated in a cyclic mode with the following steps: 1) Catalyst oxidation/regeneration with air with a start temperature of 650° C. for 10 minutes. 2) Purge the catalyst bed with nitrogen at 650° C. for 3 minutes. 3) Reduce the catalyst with hydrogen with a start temperature of 650° C. for 3 minutes. 4) Catalyst bed cooling under nitrogen from 650° C. to 585° C. and maintaining at 585° C. for 15 minutes. 5) Isobutane dehydrogenation with a start temperature of 585° C. for 10 minutes. Reactor outlet gas composition analysis with gas chromatograph at 9th minute from the start of the isobutane feed. Because the fresh catalyst activity changes during the initial cycles the catalysts were equilibrated under cyclic oxidation (air, 2 minutes)-purge (nitrogen, 4 minutes)-reduction (hydrogen, 2 minutes) conditions to achieve stable catalyst performance. The equilibration of the fresh catalyst was conducted at 650° C. for 100 cycles. Then Steps 1 to 5 were repeated for 30 cycles and the catalyst performance results after catalyst stabilization is provided in Table 2 (21-30 cycle average). The results shown in Table 2 demonstrate that the catalyst prepared according to the inventive method (Example 2) is characterized by similar performance in comparison with the catalyst prepared by the by the comparative method of Example 1.

TABLE 2
Catalyst Performance Results

| | Isobutane | Isobutylene | Isobutylene |
|---|---|---|---|
| Example | Conversion (mol % | Selectivity (mol %) | Yield (mol %) |
| 1 | 45.3 | 95.4 | 43.2 |
| 2 | 45.7 | 94.2 | 43.0 |

When ranges are disclosed herein, ranges from any lower limit may be combined with any upper limit to recite a range not explicitly recited, as well as ranges from any lower limit may be combined with any other lower limit to recite a range not explicitly recited. In the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

In the context of the present invention, at least sixteen embodiments are now described. Embodiment 1 is a method for making an alkane dehydrogenation catalyst. The method includes the steps of mixing a plurality of aluminum hydroxides, a water insoluble chromium (III) oxide source, and an alkali metal oxide source with an aqueous metal free acid solution to form a moldable mixture, the plurality of aluminum hydroxides containing from about 60 wt. % to about 99 wt. % of crystalline aluminum trihydroxide and from about 1 wt. % to about 40 wt. % of crystalline aluminum oxide-hydroxide or gelatinous aluminum hydroxide, or combinations thereof; extruding the moldable mixture to form extrudates; and drying and calcining the extrudates to produce the alkane dehydrogenation catalyst. Embodiment 2 is the method of embodiment 1, wherein the plurality of aluminum hydroxides contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof. Embodiment 3 is the method of embodiment 1, wherein the crystalline aluminum trihydroxide is one or more of bayerite and nordstrandite. Embodiment 4 is the method of embodiment 1, wherein the crystalline aluminum oxide-hydroxide is boehmite. Embodiment 5 is the method of embodiment 1, wherein the gelatinous aluminum hydroxide is one or more of amorphous aluminum hydroxide or pseudoboehmite. Embodiment 6 is the method of embodiment 1, wherein the extrudates are calcined at a temperature ranging from about 700° C. to about 1000° C. Embodiment 7 is the method of embodiment 1, wherein the alkane dehydrogenation catalyst comprises from about 60 wt. % to about 90 wt. % of alumina, from about 10 wt. % to about 40 wt. % of chromium (III) oxide, and from about 0.1 wt. % to about 5 wt. % of the alkali metal oxide.

Embodiment 8 is a method for dehydrogenation of an alkane. The method includes the steps of loading a reactor with an alkane dehydrogenation catalyst produced by: mixing a plurality of aluminum hydroxides, a water insoluble chromium (III) oxide source, and an alkali metal oxide source with an aqueous metal free acid solution to form a moldable mixture, the plurality of aluminum hydroxides containing from about 60 wt. % to about 99 wt. % of crystalline aluminum trihydroxide and from about 1 wt. % to about 40 wt. % of crystalline aluminum oxide-hydroxide or gelatinous aluminum hydroxide or combinations thereof; extruding the moldable mixture to form extrudates; and drying and calcining the extrudates to produce the alkane dehydrogenation catalyst; and supplying a feed containing alkanes through the reactor at a temperature sufficient to dehydrogenate the alkanes. Embodiment 9 is the method of embodiment 8, wherein the plurality of aluminum hydroxides contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof. Embodiment 10 is the method of embodiment 8, wherein the alkane dehydrogenation catalyst comprises from about 60 wt. % to about 90 wt. % of alumina, from about 10 wt. % to about 40 wt. % of chromium (III) oxide, and from about 0.1 wt. % to about 5 wt. % of the alkali metal oxide. Embodiment 11 is the method of embodiment 8, wherein the crystalline aluminum trihydroxide is one or more of bayerite and nordstrandite. Embodiment 12 is the method of embodiment 8, wherein the crystalline aluminum oxide-hydroxide is boehmite. Embodiment 13 is the method of embodiment 8, wherein the gelatinous aluminum hydroxide is one or more of amorphous aluminum hydroxide or pseudoboehmite. Embodiment 14 is the method of embodiment 8, wherein the extrudates are calcined at a temperature ranging from about 700° C. to 1000° C. Embodiment 15 is the method of embodiment 8, wherein the temperature sufficient to dehydrogenate alkanes ranges from 400° C. to 800° C. Embodiment 16 is the method of embodiment 8, further comprising separating dehydrogenated product from unreacted alkanes.

Other objects, features and advantages of the disclosure will become apparent from the foregoing detailed description. It should be understood, however, that the detailed description, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

1. A method for making an alkane dehydrogenation catalyst, the method comprising:
mixing a plurality of aluminum hydroxides, a water insoluble chromium (III) oxide source, and an alkali metal oxide source with an aqueous metal free acid solution to form a moldable mixture, the plurality of aluminum hydroxides containing from about 60 weight percent (wt. %) to about 99 wt. % of crystalline aluminum trihydroxide and from about 1 wt. % to about 40 wt. % of crystalline aluminum oxide-hydroxide or gelatinous aluminum hydroxide or combinations thereof;
extruding the moldable mixture to form extrudates; and
drying and calcining the extrudates to produce the alkane dehydrogenation catalyst.

2. The method of claim 1, wherein the plurality of aluminum hydroxides contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof.

3. The method of claim 1, wherein the crystalline aluminum trihydroxide is one or more of bayerite and nordstrandite.

4. The method of claim 1, wherein the crystalline aluminum oxide-hydroxide is boehmite.

5. The method of claim 1, wherein the gelatinous aluminum hydroxide is one or more of amorphous aluminum hydroxide or pseudoboehmite.

6. The method of claim 1, wherein the extrudates are calcined at a temperature ranging from about 700 degrees Celsius (° C.) to about 1000° C.

7. The method of claim 1, wherein the alkane dehydrogenation catalyst comprises from about 60 wt. % to about 90 wt. % of alumina, from about 10 wt. % to about 40 wt. % of chromium (III) oxide, and from about 0.1 wt. % to about 5 wt. % of the alkali metal oxide.

8. A method for dehydrogenation of an alkane, the method comprising:
loading a reactor with an alkane dehydrogenation catalyst produced by:
mixing a plurality of aluminum hydroxides, a water insoluble chromium (III) oxide source, and an alkali metal oxide source with an aqueous metal free acid solution to form a moldable mixture, the plurality of aluminum hydroxides containing from about 60 weight percent (wt. %) to about 99 wt. % of crystalline aluminum trihydroxide and from about 1 wt. % to about 40 wt. % of crystalline aluminum oxide-hydroxide or gelatinous aluminum hydroxide or combinations thereof;
extruding the moldable mixture to form extrudates; and
drying and calcining the extrudates to produce the alkane dehydrogenation catalyst; and
supplying a feed containing alkanes through the reactor at a temperature sufficient to dehydrogenate the alkanes.

9. The method of claim 8, wherein the plurality of aluminum hydroxides contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof.

10. The method of claim 8, wherein the alkane dehydrogenation catalyst comprises from about 60 wt. % to about 90 wt. % of alumina, from about 10 wt. % to about 40 wt. % of chromium (III) oxide, and from about 0.1 wt. % to about 5 wt. % of the alkali metal oxide.

11. The method of claim 8, wherein the crystalline aluminum trihydroxide is one or more of bayerite and nordstrandite.

12. The method of claim 8, wherein the crystalline aluminum oxide-hydroxide is boehmite.

13. The method of claim 8, wherein the gelatinous aluminum hydroxide is one or more of amorphous aluminum hydroxide or pseudoboehmite.

14. The method of claim 8, wherein the extrudates are calcined at a temperature ranging from about 700° C. to 1000° C.

15. The method of claim 8, wherein the temperature sufficient to dehydrogenate alkanes ranges from 400° C. to 800° C.

16. The method of claim 8, further comprising separating dehydrogenated product from unreacted alkanes.
